(19) United States
(12) Patent Application Publication
(10) Pub. No.: US 2013/0165965 A1
(43) Pub. Date: Jun. 27, 2013

(54) PRESSURE TRANSDUCER EQUIPPED CARDIAC PLUG

(71) Applicants: Mark Carlson, Calabasas, CA (US); Gene A. Bornzin, Simi Valley, CA (US); Timothy A. Fayram, Gilroy, CA (US)

(72) Inventors: Mark Carlson, Calabasas, CA (US); Gene A. Bornzin, Simi Valley, CA (US); Timothy A. Fayram, Gilroy, CA (US)

(73) Assignee: PACESETTER, INC., Sylmar, CA (US)

(21) Appl. No.: 13/333,793

(22) Filed: Dec. 21, 2011

(51) Int. Cl. A61B 17/03

(52) U.S. Cl. 606/213

(57) ABSTRACT

Disclosed herein is a pressure sensing left atrial occluding implantable medical device. The implantable medical device includes a cardiac plug and a micro electro-mechanical system (“MEMS”). The cardiac plug includes an expandable lobe and an expandable disc proximal the lobe. The expandable lobe is configured to expand into an anchoring arrangement within the left atrial appendage. The expandable lobe is configured to expand into an occluding arrangement with the left atrial appendage. The MEMS is coupled to the cardiac plug proximal of the disc. The MEMS is configured to sense surrounding fluid pressure.

### FIELD OF THE INVENTION

Aspects of the present invention relate to medical apparatus and methods. More specifically, the present invention relates to implantable pressure transducers and methods of manufacturing and implanting such devices.

### BACKGROUND OF THE INVENTION

Patients with congestive heart failure (“CHF”) can benefit from left atrial pressure monitoring. Unfortunately, it can be difficult to deliver and implant pressure sensing devices in the left atrium.

There is a need in the art for implantable devices capable of monitoring left atrial pressure. There is also a need in the art for methods of implanting a pressure monitoring device in the left atrium.

### BRIEF SUMMARY OF THE INVENTION

A first embodiment of the present disclosure may take the form of a pressure sensing left atrial occluding implantable medical device. The implantable medical device includes a cardiac plug and a micro electro-mechanical system (“MEMS”). The cardiac plug includes an expandable lobe and an expandable disc proximal the lobe. The expandable lobe is configured to expand into an anchoring arrangement within the left atrial appendage. The expandable lobe is configured to expand into an occluding arrangement with the left atrial appendage. The MEMS is coupled to the cardiac plug proximal of the disc. The MEMS is configured to sense surrounding fluid pressure. Depending on the embodiment, the MEMS may include a CardioMEMS as manufactured by CardioMEMS. Also, depending on the embodiment, the cardiac plug may include an AMPLATZER® Cardiac Plug as manufactured by AGA Medical Corporation.

A second embodiment of the present disclosure may take the form of a system for sensing pressure in a left atrium near the confines of a left atrial appendage. The system includes an anchor, a MEMS, and a telemetry device. The anchor is configured to achieve an anchoring interference fit within the confines of the left atrial appendage. The MEMS is coupled to the anchor and configured to protrude into the left atrium when the anchor has achieved the anchoring interference fit. The MEMS is configured to sense surrounding fluid pressure. The telemetry device is configured to wirelessly communicate with the MEMS to read fluid pressures sensed by the MEMS.

A third embodiment of the present disclosure may take the form of a method of establishing a pressure sensing arrangement for sensing left atrial pressure, the method including: creating an interference fit between an expandable anchor and a left atrial appendage; and supporting a MEMS off of the anchor such that the MEMS is located in a volume of the left atrium, the MEMS being configured to sense surrounding fluid pressure.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 is a side view of a cardiac plug.

FIG. 2 is a plan view of a micro electro-mechanical system configured for use as an implantable cardiac pressure sensor.

FIG. 3 is a plan view of a support frame.

FIG. 4 is a plan view of the micro electro-mechanical system of FIG. 2 mounted on the support frame of FIG. 3 to form a MEMS assembly.

FIG. 5 is a side view of a pressure sensor equipped cardiac plug as made possible via the coupling of the MEMS assembly of FIG. 4 to the cardiac plug of FIG. 1.

FIG. 6 depicts an embodiment of a delivery system for delivering and implanting the pressure sensor equipped cardiac plug of FIG. 5 into the left atrial appendage.

FIG. 7 is a flow chart outlining a method of delivering and implanting the pressure sensor equipped cardiac plug of FIG. 5 into the left atrial appendage.

FIG. 8 is a longitudinal cross section of the cardiac plug of FIG. 1 being loaded into the loader of the delivery system of FIG. 6.

FIG. 9 is a longitudinal cross section of the pressure sensor equipped cardiac plug of FIG. 5 within the loader and hemostasis valve of the delivery system of FIG. 6.

FIG. 10 is a longitudinal cross section of the left atrial appendage with the pressure sensor equipped cardiac plug of FIG. 5 beginning to exit the distal end of the sheath to deploy in the left atrial appendage.

FIG. 11 is the same view of the left atrial appendage as provided in FIG. 10, except the sheath has been withdrawn and the pressure sensor equipped cardiac plug is deployed in the left atrial appendage.

FIG. 12 is the same view of the left atrial appendage as provided in FIG. 11, except the delivery cable is removed and the pressure sensor equipped cardiac plug is fully implanted in the left atrial appendage.

FIG. 13 is the same view as FIG. 12, except illustrating a system for monitoring left atrial pressure.

### DETAILED DESCRIPTION

Implementations of the present disclosure involve implantable pressure sensing devices and systems for, and methods of, delivering and implanting such devices into the left atrium to allow left atrial pressure sensing. Some of the systems disclosed herein employ a micro electro-mechanical system (“MEMS”) mounted on a cardiac plug, wherein the micro electro-mechanical system is configured to sense surrounding fluid (e.g., blood) pressure, and the cardiac plug is configured to anchor in and occlude the left atrial appendage.

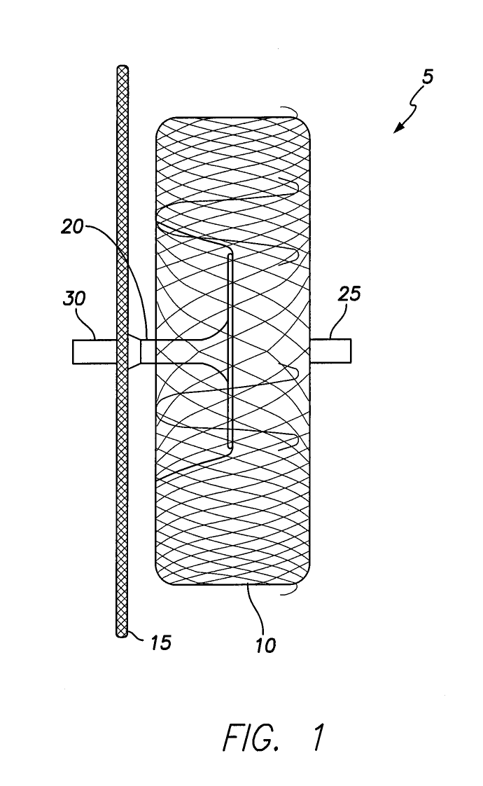

FIG. 1 is a side view of a cardiac plug 5 configured for non surgical occlusion of the left atrial appendage. In one embodiment, the cardiac plug 5 includes an expandable anchor lobe 10, an occluding disc 15 proximal the lobe 10, a waist 20 coupling the lobe 10 and disc 15 together, a distal threaded female attachment 25 distally projecting from a distal face of the lobe 10, and a proximal threaded female attachment 30 proximally extending from a proximal face of the disc 15. The lobe 10 is formed of a Nitinol wire mesh or other shape memory material such that the lobe 10 can expand to the configuration illustrated in FIG. 1 after being caused to exit the confines of a delivery device (e.g., sheath or catheter). Thus, when the plug 5 is delivered into the left atrial appendage and allowed to fully expand therein, the lobe 10 can bias against the wall surface of the left atrial appendage to anchor the plug 5 into position in the left atrial appendage. The disc 15 is also configured to self-expand upon exiting a delivery device (e.g., sheath or catheter) such that when the plug 5 is located in the left atrial appendage and anchored in place by the lobe 10, the disc 15 can bias into contact with the wall surface of the left atrial appendage to seal the left atrial appendage off from the rest of the left atrium.

In one embodiment, the cardiac plug 5 is an AMPLATZER® Cardiac Plug as manufactured by AGA Medical Corporation, which is part of the Cardiology Division of St. Jude Medical. In other embodiments, the plug 5 may be a cardiac plug as manufactured or yet to be manufactured by another entity.

The plug 5 can provide a minimally invasive mechanism for non-surgical occlusion of the left atrial appendage. When patients have atrial fibrillation (“AF”) blood stagnates in the left atrial appendage. The stagnation increases the likelihood of the blood clotting and forming a thrombus that can embolize and lead to a stroke. Usually patients are given anticoagulants which are often associated with adverse side effects including a risk of severe bleeding (0.9% to 2.7%/per year), bruising, and bleeding from the nose, gums, or GI tract. Patients need to be carefully managed with frequent blood tests to measure the normalized prothrombin time.

The plug 5 may be used to prophylactically avoid formation of thrombus in the left atrial appendage. The plug lobe 10 may be made of flexible braided Nitinol mesh. The plug disc 15 covers the “pocket” formed by the left atrial appendage.

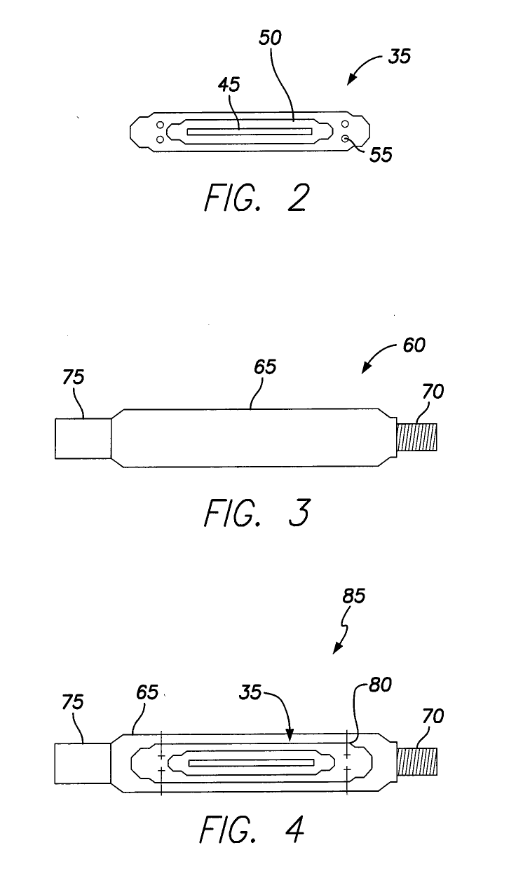

FIG. 2 is a plan view of a MEMS 35 configured for use as an implantable cardiac pressure sensor. In one embodiment, the MEMS 35 includes a coil 40 and a capacitor 45 encased in a glass enclosure 50. The MEMS 35 is capable of sensing the pressure of a fluid in which the MEMS 35 is located. Thus, when the MEMS 35 is implanted in a chamber of a patient's heart, the MEMS 35 can sense the pressure of the blood occupying the heart chamber and surrounding the MEMS 35. The MEMS 35 is further configured to wirelessly transmit its pressure readings to a telemetry wand that can be brought into close proximity to the MEMS 35 to communicate with the MEMS 35.

In one embodiment, the MEMS 35 is a CardioMEMS (“CMEMS”) as manufactured by CardioMEMS, Inc. of 387 Technology Circle NW, Suite 500, Atlanta, Ga. 30313. In other embodiments, the MEMS 35 may be a CMEMS as manufactured or yet to be manufactured by another entity.

As indicated in FIG. 2, the glass enclosure 50 of the MEMS 35 has holes 55 for attachment of a support or stabilization frame 60, which is shown in plan view in FIG. 3. As indicated in FIG. 3, the frame 60 includes a platform portion 65, a threaded male member 70 distally projecting from the platform portion 65, and a threaded female member 75 proximally projecting from the platform portion 65.

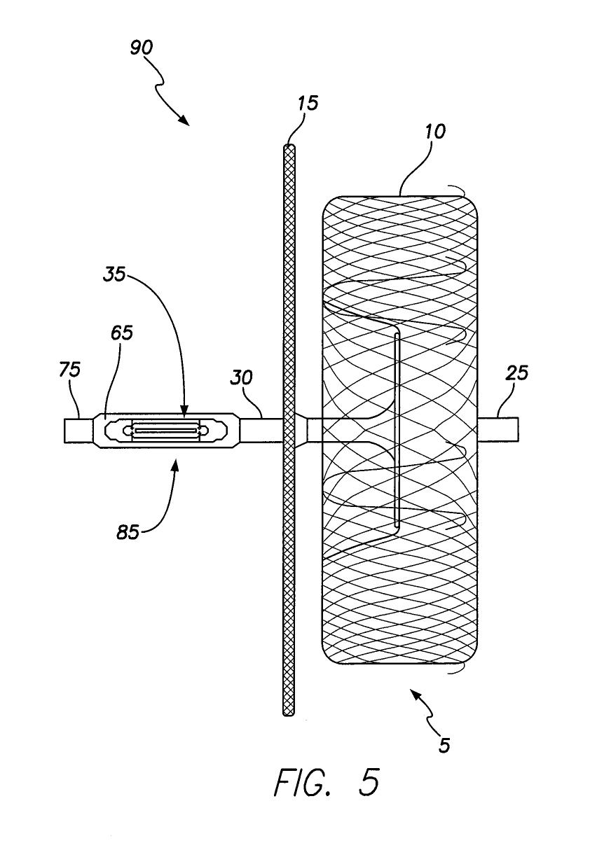

FIG. 4 is a plan view of the MEMS 35 mounted on the support frame 60. As illustrated in FIG. 4, in one embodiment, the MEMS 35 is located on the platform portion 65 between the two threaded members 70, 75. The platform portion 65 may be in the form of a metallic frame that surrounds the MEMS 35 mounted thereon. The support frame 60 may be made of a biocompatible, biostable metal like titanium, titanium alloy or stainless steel. Stainless steel wires 80 extend through the holes 55 and may be laser welded or otherwise secured to the platform portion 65. Alternatively, the MEMS 35 may be secured to the platform portion 65 via other arrangements such as, for example, a biocompatible adhesive. With the MEMS 35 secured to the support frame 60 as indicated in FIG. 4, the resulting assembly 85 can be considered to be a MEMS assembly 85 that can be removably coupled to the cardiac plug 5 as will now be discussed with respect to FIG. 5.

FIG. 5 is a side view of a pressure sensor equipped cardiac plug 90 as made possible via the coupling of the MEMS assembly 85 of FIG. 4 to the cardiac plug 5 of FIG. 1. As can be understood from FIG. 5, the distal threaded male member 70 of the MEMS assembly 85 is threadably received in the proximal threaded female attachment 30 of the plug 90. Thus, the cardiac plug 90 is equipped with a pressure sensor that will allow the left atrial pressure to be measured and reported for the duration of the plug 90 being implanted in the left atrial appendage.

Depending on the embodiment, the MEMS assembly 85 may be coupled to the plug 5 at some date after implant of the plug 5, immediately after the plug is implanted, or as a combined implant process in which the MEMS assembly 85 and plug 5 are secured together to form the pressure sensor equipped cardiac plug 90 as shown in FIG. 5 and then implanted as a whole or complete device 90.

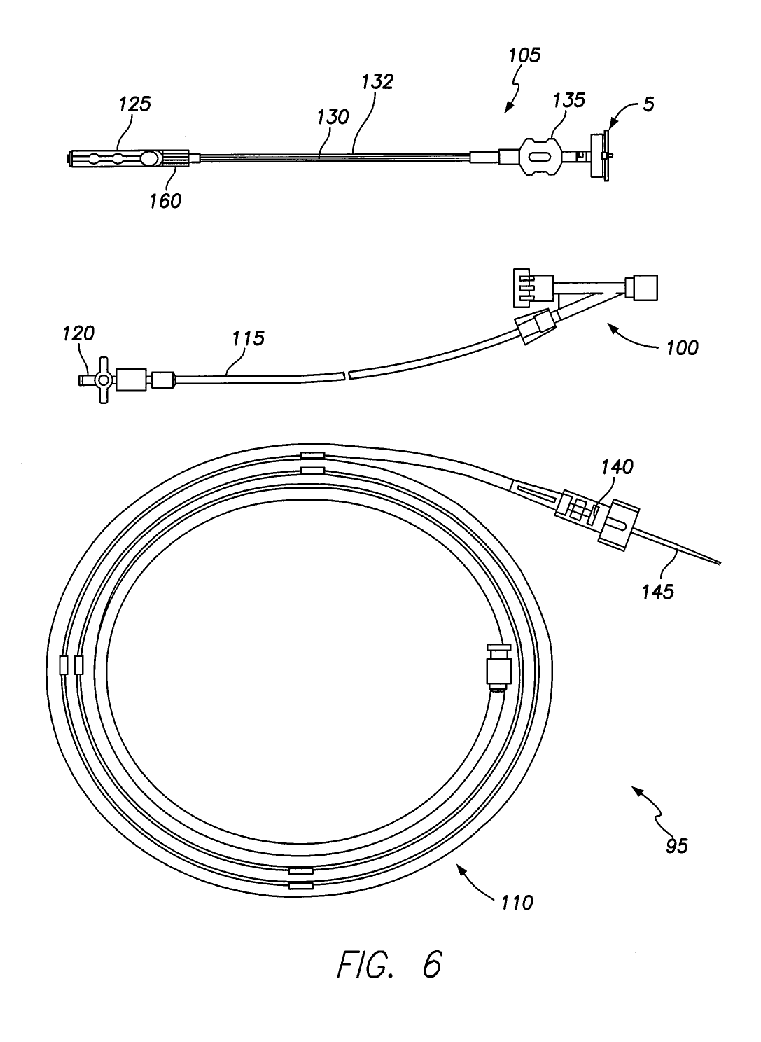

FIG. 6 depicts an embodiment of a delivery system 95 for delivering and implanting the pressure sensor equipped cardiac plug 90 of FIG. 5 into the left atrial appendage. As illustrated in FIG. 6, the delivery system 95 may include a hemostasis valve 100, a loader 105, and a hoop dispenser 110. The hemostasis valve 100 includes a tubular extension 115 leading to a stop cock 120. The loader 105 includes a loading cable vise 125 that is coupled to a loading cable 130 within a tube 132 extending to a hub 135. The hoop dispenser 110 includes a delivery cable device 140 and a delivery cable 145. The delivery system 95 may be provided in the form of a medical or surgical kit complete with instructions provided on or in the packaging enclosing the delivery system or separate from the kit in the form of hardcopy instructions or instructions available via the internet. The kit may further include the MEMS assembly 85 and the cardiac plug 5 in the same packaging or separately packaged.

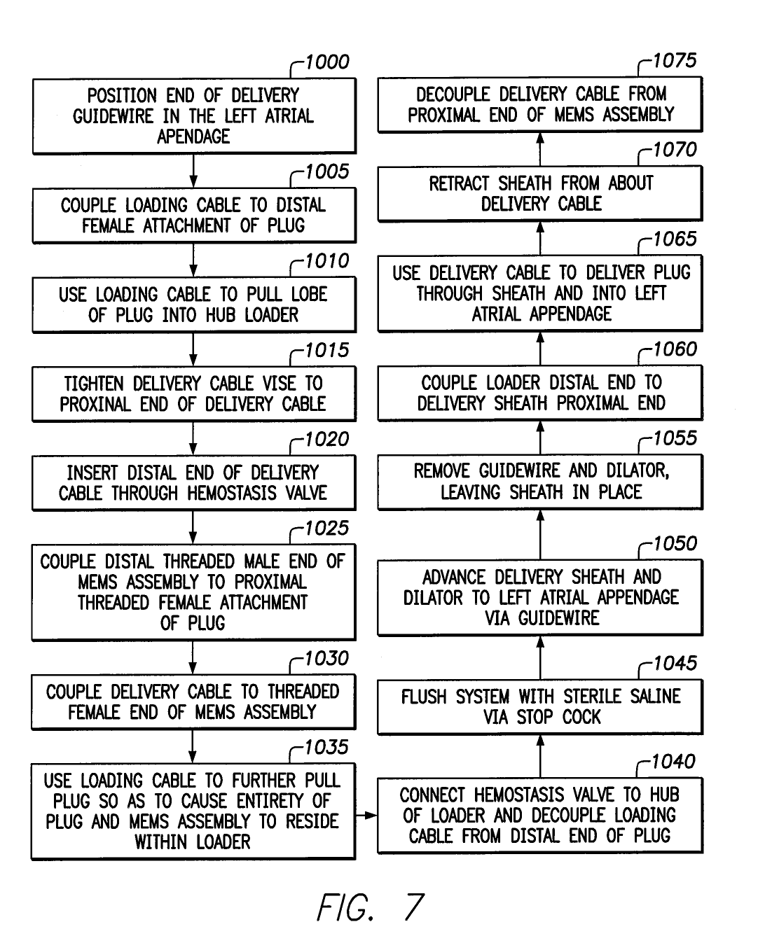

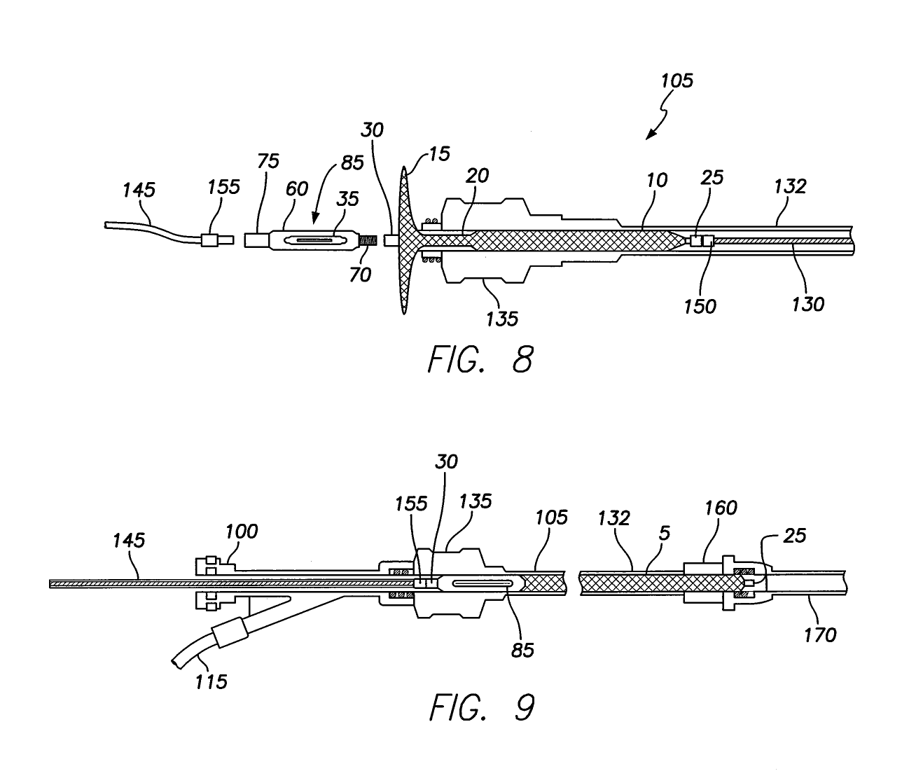

To begin a discussion of a method of delivering and implanting the pressure sensor equipped cardiac plug 90 of FIG. 5 via the delivery system 95 of FIG. 6, reference is now made to FIG. 7, which is a flow chart outlining the method. A delivery guidewire (not shown) is negotiated through the vascular and cardiac structures until the distal end of the guidewire is located in the left atrial appendage [block 1000]. As shown in FIG. 8, which is a longitudinal cross section of the cardiac plug 5 being loaded into the loader 105, a threaded male attachment 150 of the loading cable 130 is threaded into the distal female attachment 25 of the plug 5 to couple the loading cable 130 to the plug 5 [block 1005]. The loading cable vise 125 (shown in FIG. 6) is pulled so as to cause the loading cable 130 to pull the plug 5 until the lobe 10 of the plug 5 is fully retracted within the hub 135 of the loader 105, but not so far as to cause the disc 15 to be recaptured in the loader hub 135 [block 1010]. The delivery cable 145 is removed from the hoop dispenser 110 and the delivery cable vise 140 is tightened to the proximal end of the delivery cable 145 [block 1015]. The distal end of the delivery cable 145 is inserted through the hemostasis valve 100 [block 1020].

As can be understood from FIG. 8, the distal threaded male member 70 of the MEMS assembly 85 is tightly threaded into the proximal threaded female attachment 30 of the plug 5 to securely couple the MEMS assembly 85 to the plug 5 [block 1025]. In some embodiments, the MEMS assembly 85 may be welded, adhered, or otherwise permanently attached to the proximal end 30 of the plug 5 such that the MEMS assembly 85 and plug 5 come from the manufacturer as a completely assembled, integral pressure sensor equipped cardiac plug 90. In such and embodiment, the physician could decide to enable the MEMS 35 or simply not enable the MEMS 35, the plug 5 just serving as an occluding device. The distal threaded male attachment 155 of the delivery cable 145 is loosely threaded into the proximal threaded female member 75 of the MEMS assembly 85 to couple the delivery cable to the MEMS assembly 85 in such a manner to allow the delivery cable 145 to be easily decoupled from the member 75 upon delivery and implantation of the pressure sensor equipped cardiac plug 90 into the left atrial appendage [block 1030].

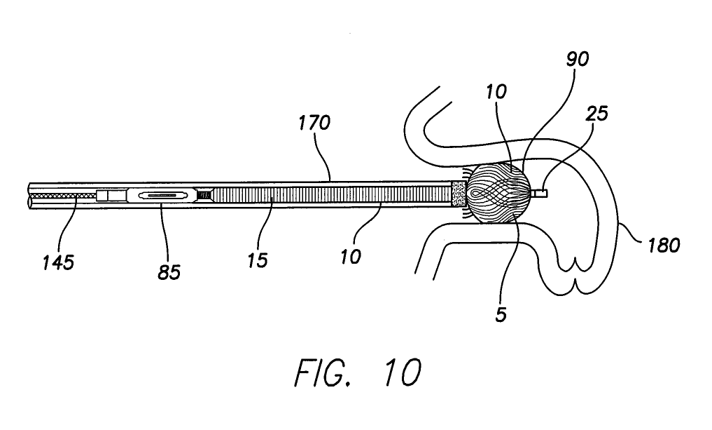

At this point in the method, the loading cable 130 can be further pulled to recapture the disc 15 of the plug 5 and the MEMS assembly 85 into the loader 105 such that the distal threaded male attachment 155 of the delivery cable 145 resides just within the proximal end of the hub 135 of the loader 105 [block 1035], as illustrated in FIG. 9, which is a longitudinal cross section of the pressure sensor equipped cardiac plug 90 within the loader 105 and hemostasis valve 100. The hemostasis valve 100 is then connected to the hub 135 of the loader 105 and the loading cable is decoupled from the distal end 25 of the plug 5 [block 1040]. The stop cock 120 of the hemostasis valve 100 is coupled to a sterile saline supply and the sterile saline is caused to flow until the sterile saline exits both ends of the assembly of the loader 105 and hemostasis valve 100 [block 1045]. A drip with Heparin may be run through the stop cock to keep the delivery sheath or catheter patent during delivery and implantation.

A delivery sheath and dilator are advanced into the patient over the guidewire until the distal end of the sheath is located in the area of the left atrial appendage to be occluded by the plug 5 [block 1050]. The guidewire and dilator are removed from the patient, leaving the delivery sheath in place [block 1055]. After further flushing of the assembly of the loader 105 and hemostasis valve 100 with sterile saline, the distal end 160 of the loader 135 is coupled to the proximal end 165 of the delivery sheath 170 [block 1060], as depicted in FIG. 9. The delivery cable 145 is used to distally advance the pressure sensor equipped cardiac plug 90 of FIG. 5 through the sheath 170 to cause the pressure sensor equipped cardiac plug 90 to exit the sheath distal end 175 in the left atrial appendage 180 [block 1065], as depicted in FIG. 10, which is a longitudinal cross section of the left atrial appendage 180. Once the pressure sensor equipped cardiac plug 90 is deployed in the left atrial appendage 180, the delivery cable 145 can be held in place as the sheath 170 is retracted from about the delivery cable 145 [block 1070].

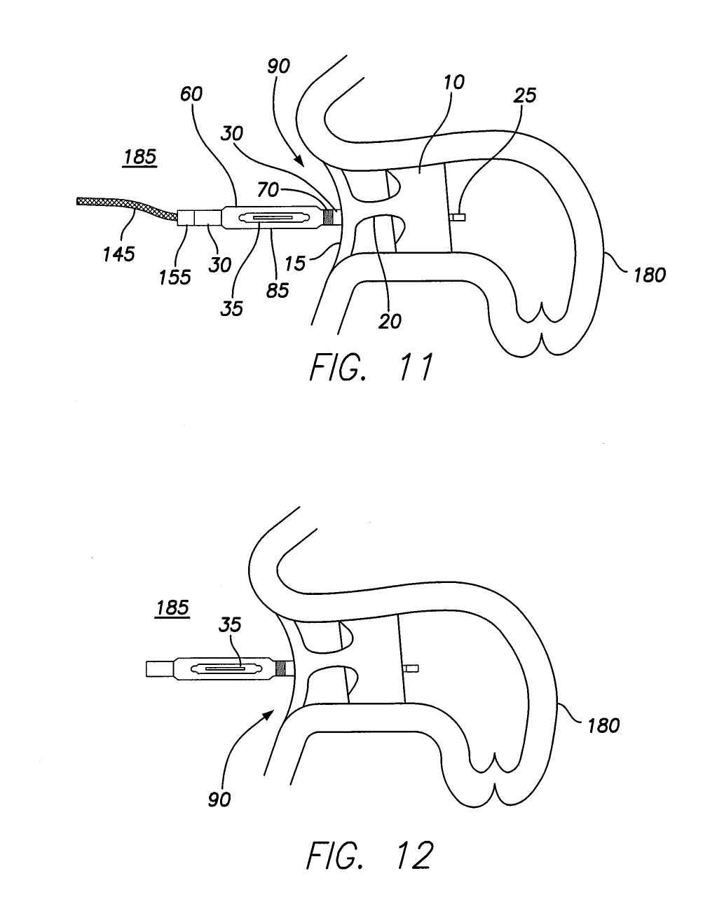

As can be understood from FIG. 11, which is the same view of the left atrial appendage as FIG. 10, the pressure sensor equipped cardiac plug 90 is positioned to occlude the left atrial appendage via the disc 15 extending across the opening into the left atrial appendage 180. Also, the lobe 10 is fully expanded within the confines of the left atrial appendage 180 to anchor the pressure sensor equipped cardiac plug 90 within the left atrial lobe 180, and the lobe 10 is generally parallel with the longitudinal axis of the left atrial appendage.

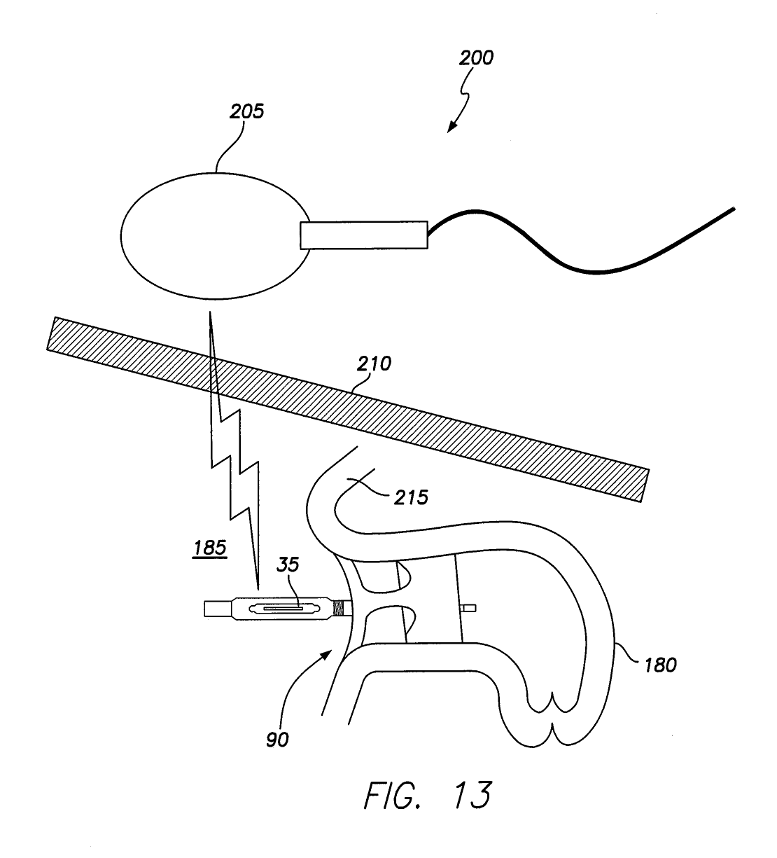

As illustrated in FIG. 12, delivery cable 145 is decoupled from the proximal end of the MEMS assembly 85 once the pressure sensor equipped cardiac plug 90 is fully deployed in the left atrial appendage 180 as desired [block 1075]. As depicted in FIG. 12, the MEMS assembly 85 projects into the left atrium volume 185 once the pressure sensor equipped cardiac plug 90 is anchored in and occludes the left atrial appendage 180. As a result of its position within the left atrium volume 185, the MEMS 35 can sense the blood pressure in the left atrium volume 185 and report the sensed blood pressure to a telemetry wand brought in close proximity to the patient's chest region over the left atrium.

As can be understood from FIG. 13, which is the same view as FIG. 12, in one embodiment, the MEMS 35 and anchoring lobe 35 may be part of a system 200 for sensing pressure in a left atrium 185 near the confines of a left atrial appendage 180. For example, the system 200 may include an anchor (e.g., the cardiac plug 5 or simply the lobe 10 itself), a MEMS 35, and a telemetry device 205. The anchor 10 is configured to achieve an anchoring interference fit within the confines of the left atrial appendage 180. The MEMS 35 is coupled to the anchor 10 and configured to protrude into the left atrium 185 when the anchor 10 has achieved the anchoring interference fit. The MEMS 35 is configured to sense surrounding fluid pressure. The telemetry device 205 is configured to wirelessly communicate through the patient chest wall 210, heart wall 215 and other surrounding tissue with the MEMS 35 to read fluid pressures sensed by the MEMS 35.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. A pressure sensing left atrial occluding implantable medical device comprising:
a cardiac plug comprising an expandable lobe and an expandable disc proximal the lobe, wherein the expandable lobe is configured to expand into an anchoring arrangement within the left atrial appendage, and the expandable lobe is configured to expand into an occluding arrangement with the left atrial appendage; and
a MEMS coupled to the cardiac plug proximal of the disc, wherein the MEMS is configured to sense surrounding fluid pressure.

2. The device of claim 1, wherein the MEMS includes a CardioMEMS as manufactured by CardioMEMS, Inc.

3. The device of claim 1, wherein the cardiac plug includes an AMPLATZER® Cardiac Plug as manufactured by AGA Medical Corporation.

4. The device of claim 1, wherein the expandable lobe includes a Nitinol mesh.

5. The device of claim 1, wherein the device further includes a platform secured to the cardiac plug and on which the MEMS is secured.

6. The device of claim 5, wherein the platform includes titanium.

7. The device of claim 1, wherein the platform includes a distal threaded male member and a proximal threaded female member.

8. The device of claim 7, wherein the cardiac plug further comprises a proximal female threaded attachment that threadably couples with the distal threaded male member.

9. The device of claim 5, wherein the platform on which the MEMS is secured is permanently attached to the cardiac plug.

10. The device of claim 5, wherein the MEMS is secured to the platform via wires.

11. A system for sensing pressure in a left atrium near the confines of a left atrial appendage, the system comprising:
an anchor configured to achieve an anchoring interference fit within the confines of the left atrial appendage;
a MEMS coupled to the anchor and configured to protrude into the left atrium when the anchor has achieved the anchoring interference fit, wherein the MEMS is configured to sense surrounding fluid pressure; and
a telemetry device configured to wirelessly communicate with the MEMS to read fluid pressures sensed by the MEMS.

12. The system of claim 11, wherein the anchor is part of a cardiac plug.

13. The system of claim 12, wherein at least one of the MEMS includes a CardioMEMS as manufactured by CardioMEMS, Inc. or the cardiac plug includes an AMPLATZER® Cardiac Plug as manufactured by AGA Medical Corporation.

14. The system of claim 11, wherein the anchor includes an expandable lobe that is configured to achieve the anchoring interference fit.

15. The system of claim 14, wherein the lobe includes a Nitinol mesh.

16. A method of establishing a pressure sensing arrangement for sensing left atrial pressure, the method comprising:
creating an interference fit between an expandable anchor and a left atrial appendage; and
supporting a MEMS off of the anchor such that the MEMS is located in a volume of the left atrium, the MEMS being configured to sense surrounding fluid pressure.

17. The method of claim 16, wherein the MEMS and anchor are coupled together before being delivered together to the left atrial appendage.

18. The method of claim 16, wherein the MEMS is configured to wirelessly communicate with a telemetry wand.

19. The method of claim 16, wherein the anchor is part of a cardiac plug.

20. The method of claim 19, wherein at least one of the MEMS includes a CardioMEMS as manufactured by CardioMEMS, Inc. or the cardiac plug includes an AMPLATZER® Cardiac Plug as manufactured by AGA Medical Corporation.
